(19) United States
(12) Patent Application Publication
(10) Pub. No.: US 2013/0184685 A1
(43) Pub. Date: Jul. 18, 2013

(54) METHOD FOR PREPARING RED BLOOD CELLS FOR INFUSION

(71) Applicants: Pierre Buffet, Paris (FR); Guillaume Deplaine, Guyancourt (FR); Innocent Safeukui-Noubissi, Paris (FR)

(72) Inventors: Pierre Buffet, Paris (FR); Guillaume Deplaine, Guyancourt (FR); Innocent Safeukui-Noubissi, Paris (FR)

(73) Assignees: ASSISTANCE PUBLIQUE - HOPITAUX DE PARIS, PARIS (FR); UNIVERSITE PIERRE ET MARIE CURIE (PARIS 6), PARIS (FR)

(21) Appl. No.: 13/810,513

(22) PCT Filed: Jul. 20, 2011
(86) PCT No.: PCT/EP2011/062442

§ 371 Date: Mar. 29, 2013

(30) Foreign Application Priority Data
Jul. 20, 2010 (EP) 10305801.2
Dec. 3, 2010 (EP) 10306352.5

(51) Int. Cl. A61M 1/34

(52) U.S. Cl. CPC A61M 1/34
USPC 604/522; 210/807

(57) ABSTRACT

The present invention relates to methods to prepare red blood cells (RBCs) for infusion to a patient. Depletion of RBCs with altered deformability is achieved by filtration through micro-bead filters.

### FIELD OF THE INVENTION

The present invention concerns a method to prepare red blood cells (RBCs) to infuse a patient, and in particular, a method to deplete RBCs with altered deformability from blood product to be infused. The present invention also concerns a method for rejuvenation of stored RBC containing blood product for transfusion. It also concerns a method for treating an inherited or acquired RBC disorder.

### BACKGROUND OF THE INVENTION

Blood transfusions may be required in various situations such as massive blood loss due to trauma or surgery, severe anaemia, or for people suffering from hemophilia or sickle-cell disease. Before transfusion, red blood cells (RBCs) may be stored in blood banks for up to 42 days under controlled conditions. However, several studies showed a correlation between RBC storage duration and morbidity and mortality rates after transfusion. In particular, an increased risk of post-operative complications as well as reduced short-term and long-term survival have been associated with transfusion of RBCs stored for more than 2 weeks (Koch et al., 2008). Accordingly, the use of fresh RBC is strongly recommended for critically ill patients or pediatric patients.

During their storage, RBCs undergo functional and rheological property changes. In particular, it has been demonstrated that RBC storage results in decreased RBC deformability (La Celle, 1969; Stadler, Toumbis and Ambrus, 1994; Kirkpatrick et al., 1998; Huruta et al., 1998; Izzo et al., 1999). This altered deformability might in part explain the increased incidence of infectious complications and respiratory distress in severely ill patients transfused with “older red cell concentrates” (Koch et al., 2008). Furthermore, rigid RBCs can produce capillary slugging and occlusion, leading to local ischemia.

In vivo, the spleen filters out poorly deformable RBC by retaining them upstream from narrow inter-endothelial slits in the wall of red pulp sinuses. This mechanical retention contributes to the physiological clearance of senescent RBC. However, this clearance process is also involved in the pathogenesis of inherited RBC disorders, malaria, and in complications following transfusion with long-stored blood.

RBC deformability depends on at least three factors: (i) membrane viscoelasticity, (ii) cytoplasmic viscosity, and (iii) geometry of the cell (the RBC surface to volume ratio) (Nash et al., 1989; Mohandas et al., 2008). For a population of RBCs, the deformability can be measured by several techniques including elongation under shear stress (ektacytometry, LORCA) (Mohandas et al., 2008), or time of transit through micropore filtration systems (Mohandas et al., 1979). The deformability of a RBC may be determined by using micropipettes, optical tweezers, and optical magnetic testing cytometry (OMTC) that stretch either the whole RBC or a limited area of its plasma membrane.

The limited storage time of RBCs means that blood centers have often difficulties to maintain even a few day supply for routine transfusion demands and cannot have a stockpile of blood to prepare for a disaster. The storage time of RBCs can be extended by freezing the blood with a mixture of glycerol or by using drugs, such as Rejuvesol®, that are able to biochemically modify long-term stored red blood cells back to their “fresh” cell characteristics. However, these methods remain expensive and the use of drugs may induce side effects such as allergic reactions.

Increasing the deformability of stored RBC population before transfusion would be an efficient approach for product rejuvenation. Some drugs such as pentoxyfylline, vinpocetin and piracetam have been proposed for use in improving RBC deformability. However, these drugs are not suitable for every type of patients. For example, these drugs cannot be used for cardiac patients due to their pharmacological activities and possible adverse effects on the cardiovascular system. On another hand, filtration of RBCs has been recently described to isolate, detect or recover RBCs with abnormal deformability, and in particular RBCs infected by a Plasmodium (international patent application WO 2009/144586). However, these techniques were only used for screening or diagnosis purposes.

Consequently, there remains a strong need for cheap and safe methods to obtain RBC preparations that are suitable for infusion and which would spare blood resources while improve efficiency and tolerability of blood infusion.

### SUMMARY OF THE INVENTION

The object of the present invention is to provide methods to prepare RBCs to infuse a patient. In that context, the invention provides methods to deplete RBCs with altered deformability from blood product to be infused.

The inventors have herein demonstrated that filtration of a population of RBCs with altered deformability through filtering units comprising micro-beads not only restored the deformability of the RBC population but also preserved the morphology and the integrity of RBCs. On this basis, the invention provides methods for obtaining RBC preparation for infusing a patient and methods for rejuvenation of stored blood for transfusion.

In a first aspect, the present invention concerns a preparation of RBCs for use in infusion to a patient, wherein the RBC preparation has been obtained by filtration of RBCs through a filtering unit comprising one or several layers of micro-beads from 5 to 50 μm, preferably from 5 to 25 μm of diameter. In a particular embodiment, the RBCs that are subjected to filtration are packed RBCs stored in a blood bank for more than 3 weeks.

In another aspect, the invention concerns a method for administering RBCs to a patient, which method comprises providing RBCs, filtering said RBCs through a filtering unit comprising one or several layers of micro-beads from 5 to 50 μm, preferably from 5 to 25 μm of diameter, recovering the filtrate and infusing the patient with said filtrate.

In another aspect, the invention concerns a method for preparing RBCs for infusion to a patient, which method comprises filtering RBCs through a filtering unit comprising one or several layers of micro-beads from 5 to 50 μm, preferably from 5 to 25 μm of diameter, recovering the filtrate, and obtaining RBC depleted of rigid red blood cells.

In a further aspect, the invention concerns a method for rejuvenation of RBCs for transfusion, which method comprises filtering said RBCs through a filtering unit comprising one or several layers of micro-beads from 5 to 50 μm, preferably from 5 to 25 μm of diameters and recovering the filtrate, whereby obtaining a rejuvenated RBC preparation depleted of rigid red blood cells.

In a further aspect, the present invention also concerns a method for treating a patient having an inherited or acquired RBC disorder, which method comprises collecting blood from the patient, filtering said blood through a filtering unit comprising one or several layers of micro-beads from 5 to 50 μm, preferably from 5 to 25 μm of diameter, recovering the filtrate and infusing the patient with said filtrate.

### BRIEF DESCRIPTION OF THE DRAWINGS

FIG. 1: Schematic flow route and shape deformation of RBC squeezed through large or narrow inter-bead spaces.

FIG. 2: Filtration of rigid RBC through a bead layer. Metal beads from a single batch were purchased as calibrated mixtures of 5-15 and 15-25 μm diameter (IPS Industrie des Poudres Sphériques, Annemasse, France). A 50/50 mixture of 5-15 and 15-25 μm beads was deposited onto the anti-aerosol filter of a commercial 1000 μl tip. Decantation of beads following their introduction into an inverted 1000 μl-tip, leading to the formation of a 5 mm-thick bead layer (black arrow) above the anti-aerosol filter. RBC from a volunteer or a patient blood were re-suspended at 2% hematocrit in filtration medium (PBS/1% albumin), then introduced in the tubing upstream from the bead layer (1). An electric pump containing filtration medium was then immediately switched on (2), gently percolating RBC through the bead layer (3). Upon rinsing of the bead layer with 7 ml of filtration medium, the downstream sample was retrieved (4).

FIG. 3: Typical upstream (1)—to-downstream (2) decrease of heated (FIG. 3A), or parasitized (FIG. 3B)—RBC proportion as observed by PKH (a green fluorescent cell dye with long aliphatic tails) fluorescence or on Giemsa-stained smears.

FIG. 4: Kinetic of elongation index and retention rate of stored vs fresh RBC. Elongation index was monitored by LORCA during 6 weeks on stored and fresh RBC. Elongation index was measured before and after filtration of stored RBC (black dotted curve or black curve, respectively) and of fresh RBC (grey curve and grey dotted curve, respectively). The range of the elongation index of fresh RBC was between 0,600 and 0,640 (Light grey zone). Filtration through micro-bead device was performed in parallel and retention rate was calculated. Storage-dependent increase of retention rate was observed for stored RBC (black bar), contrasting with an absence of retention for fresh RBC (grey bar).

FIG. 5: Morphology of RBC after filtration through 2 μm polycarbonate sieves or micro-beads. As opposed to filtration through polycarbonate sieves (2 μm-wide, 20 μm-long channels), there were no schizocyte (horizontal arrows), dacryocyte (vertical arrows), anisocyte (rectangle) or poikilocyte (*) downstream from the micro-beads, indicating that the micro-bead filtration process preserves RBC morphology.

FIG. 6: Schematic representation of filtering unit systems used in Examples. FIG. 6A: Filtering system used in Example 1 and comprising 250 μL of filtering micro-beads. FIG. 6B: Filtering system used in Example 2 (infusion set/bubble trap system) and comprising 1 mL of filtering micro-beads. FIG. 6C: Filtering system used in Example 2 (“Filtron” device) and comprising 12 mL of filtering micro-beads.

FIG. 7: Retention rates of RBCs infected with Plasmodium falciparum. Rings (1), trophozoites (2) or schizonts (3) in the infusion set/bubble trap system (empty circles for each of three independent experiments, dotted line for the mean) or with the “Filtron” device (diamonds for each of two independent experiments, full line for the mean). The insert shows retention rates of RBCs infected with similar parasite stages (R: rings; T: trophozoites; S: schizonts) and filtered with the filtering system of example 1.

### DETAILED DESCRIPTION OF THE INVENTION

The present invention firstly concerns a preparation of red blood cells (RBCs) for use in infusion to a patient, wherein the RBC preparation has been obtained by filtration of RBCs through a filtering unit comprising one or several layers of micro-beads from 5 to 50 μm, preferably from 5 to 25 μm of diameter.

As used herein, the term “patient” preferably refers to a human including adult, child, infant and neonate. However, this term can also refer to non-human animals, preferably mammals such as dogs, cats, horses, cows, pigs, sheep and non-human primates.

In the present document, the terms “red blood cell”, “RBC” or “erythrocyte” can be interchangely used.

In an embodiment, the RBCs that are subjected to filtration are whole blood. The term “whole blood” refers to unseparated venous blood, i.e. containing all blood components.

In another embodiment, the RBCs that are subjected to filtration are a RBC containing fraction. The term “RBC containing fraction” refers to a fraction of blood components containing RBCs. In a particular embodiment, the RBC containing fraction is a RBC concentrate, also named packed RBCs and obtained from whole blood by removal of one or several blood components such as plasma, platelets and/or leukocytes.

The composition comprising the RBCs that are subjected to filtration may comprise one or several preservative additives such as anti-coagulant solution (e.g. citrate-dextrose-phosphate or saline-adenine-glucose-mannitol), or any other compounds used to preserve integrity of blood components and in particular of erythrocytes.

In a preferred embodiment, the hematocrit of the composition comprising the RBCs that are subjected to filtration is less than 60%, preferably in the range of 0.1% to 50%, more preferably in the range of 0.5% to 30%, and even more preferably in the range of 0.5% to 5%. The hematocrit value of the composition may be adjusted by dilution with suitable buffered solution such as, for instance, PBS, RPMI or Krebs medium supplemented with 0.5 to 2% albumin. After filtration, the filtrate may be concentrated before to be infused to the patient, for instance by centrifugation.

In an embodiment, the RBCs that are subjected to filtration may have been collected for less than 24 hours. In particular, these RBCs may originate from the recipient. They may have been collected before or during a surgery and may be administered to the recipient during or after a surgery (autologous donation).

In another embodiment, the RBCs that are subjected to filtration have been previously stored, for instance in a blood bank. As used herein, the term “stored RBCs” refers to RBCs that have been collected for more than 24 hours. As used herein, a blood bank is a bank in which RBCs, obtained by blood donations, are stored and preserved for later use in blood transfusions. In a particular embodiment, the RBCs that have been previously stored are packed RBCs or are contained in whole blood. According to storage blood product regulations, RBCs may have been stored at 1 to 6° C. during up to 35 days for whole blood and up to 42 days for packed RBCs. In a particular embodiment, RBCs have been stored during at least 3 weeks. In a preferred embodiment, the RBCs that have been previously stored are packed RBCs, and more particularly packed RBCs stored during more than 3 weeks. In a particular embodiment, the RBCs that have been previously stored are packed RBCs buffered with glycerol which has been frozen and stored during up to 3 years at −65° C.

In another embodiment, the RBCs that are subjected to filtration have been previously heated. Several studies demonstrated that heating RBCs induces a decrease of RBC deformability. However, some indications require heated RBCs such as massive blood replacement, major operations on children, operations on patients under induced hypothermia, or transfusion to hypothermic trauma patient. For instance, RBCs may have been heated during 15 min to one hour at a temperature ranging from 45 to 60° C. Preferably, RBCs have been heated during 15 min to 45 min at a temperature ranging from 45 to 55° C.

In a further embodiment, the RBCs that are subjected to filtration have been obtained from a patient having an inherited or acquired RBC disorder. The inherited or acquired RBC disorder may be a disorder inducing an altered deformability of RBCs, i.e. a decrease of the deformability, and thus an increased concentration of rigid RBC in blood. The inherited or acquired RBC disorder may be, for instance, hereditary or inherited spherocytosis, elliptocytosis (also named ovalocytosis), sepsis, hemoglobinopathy such as alpha or beta thalassemia, sickle cell disease, sickle cell trait (or sicklemia), diabetes mellitus, hemolytic anaemia such as auto immune hemolytic anaemia, and red blood cell enzyme deficiency such as glucose-6-phosphate deshydrogenase or pyruvate kinase deficiency. In a preferred embodiment, the patient is affected by sickle cell disease. The patient may also be infected by a protozoan parasite of the genus Plasmodium. Indeed, red blood cells infected by such as a parasite exhibit an increased rigidity. Uninfected red blood cells in the blood of these patients may also display an altered deformability. In particular, the parasite may be chosen from the group consisting of Plasmodium falciparum, Plasmodium ovale, Plasmodium malariae, Plasmodium knowlesi, Plasmodium inui, Plasmodium cynomolgi, Plasmodium simiovale, Plasmodium brazilianum, Plasmodium schwetzi and Plasmodium simium, preferably in the group consisting of Plasmodium falciparum, Plasmodium ovale, Plasmodium knowlesi and Plasmodium malariae, and more preferably the parasite is Plasmodium falciparum.

In a particular embodiment, the RBCs that are subjected to filtration are a RBC fraction of erythrocytapheresis. Erythrocytapheresis is an apheresis procedure by which RBCs are separated from whole blood. It is an extracorporeal blood separation method whereby whole blood is extracted from a donor or a patient, RBCs are separated, and the remaining blood (plasma, leukocyte and platelet fractions) is returned to circulation. This type of apheresis is most commonly carried out by centrifugation. Erythrocytapheresis is commonly used to remove RBCs in patients having an inherited or acquired RBC disorder, and in particular in patients having sickle cell disease. This blood separation may also be used in patients with severe malaria. According to the present invention, the RBC fraction recovered by erythrocytapheresis is filtered through a micro-beads filtering unit as disclosed herein. The resulting RBC preparation is depleted of rigid RBCs and can be infused to the patient with the other blood fractions (plasma, leukocyte and platelet fractions).

In an embodiment, the RBCs that are subjected to filtration are obtained from the patient to be infused.

In another embodiment, the RBCs that are subjected to filtration are obtained from a blood bank.

In a preferred embodiment, the RBCs that are subjected to filtration are obtained from a human.

The filtering unit comprises one or several layers of micro-beads. Any biocompatible material suitable to produce micro-beads of less than 25 μm may be used to produce the filtering unit. In a preferred embodiment, microbeads are of metallic or polymeric biocompatible materials, or of silicon, glass, cellulose, synthetic ceramic or composite biocompatible materials. Examples of preferred biocompatible metallic materials include stainless steel, tantalum, nitinol, gold, tin and titanium alloy. Examples of preferred polymeric materials include poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), polyD,L-lactide-co-caprolactone) (PLA/PCL), poly(glycolide-co-caprolactone) (PGA/PCL), polyanhydrides (PAN), poly(ortho esters), poly(phoshate ester), poly(amino acid), poly(hydroxy butyrate), polyacrylate, polyacrylamid, poly(hydroxyethyl methacrylate), polyurethane, polysiloxane and their copolymers.

Micro-beads present in the filtering unit have a diameter in the range of from 5 to 50 μm, preferably from 5 to 40 μm, still preferably from 5 to 30 μm, even preferably from 5 to 25 μm. As used herein, the term “micro-beads” refers to spherical particles or particles approximately spherical in shape. Channels formed by the inter-bead space within the filtering unit preferably varie between 0.74 and 9.4 μm, and more preferably between 1.85 μm and 9.4 μm. In a preferred embodiment, the filtering unit is constituted by a mix of micro-beads having a size distribution of 15 μm to 50 μm, preferably of 15 μm to 25 μm, diameter and micro-beads having a size distribution of 5 μm to 15 μm diameter. Preferably, the filtering unit is constituted by, or comprises at least one layer constituted by, a mix of micro-beads having a size distribution of 15 to 25 μm, or 15 to 50 μm, diameter and micro-beads having a size distribution of 5 μm to 15 μm diameter and comprises at least 1%, 5%, 10%, 15%, 20%, 25%, 30%, 35%, 40%, 45%, 50%, 55%, 60%, 70%, 75% or 80% (w/w) of micro-beads having a size distribution of 5 μm to 15 μm diameter. In a particular embodiment, the filtering unit is constituted by, or comprises at least one layer constituted by, a mix of micro-beads having a size distribution of 15 μm to 25 μm diameter and micro-beads having a size distribution of 5 μm to 15 μm diameter, said mix comprising at least 50% (w/w) of micro-beads having a size distribution of 5 μm to 15 μm diameter. In a particularly preferred embodiment, the filtering unit is constituted by, or comprises at least one layer constituted by, about 50% (w/w) of micro-beads having a size distribution of 15 μm to 25 μm diameter and about 50% (w/w) of micro-beads having a size distribution of 5 μm to 15 μm diameter. In another particularly preferred embodiment, the filtering unit is constituted by, or comprises at least one layer constituted by, about 30% (w/w) of micro-beads having a size distribution of 15 μm to 25 μm diameter and about 70% (w/w) of micro-beads having a size distribution of 5 μm to 15 μm diameter. As used in this specification, the term “about” refers to a range of values±10% of the specified value, preferably to a range of values±5% of the specified value. In another particular embodiment, the filtering unit comprises a layer of micro-beads having a size distribution of 15 μm to 25 μm diameter and a layer above comprising micro-beads having a size distribution of 5 μm to 15 μm diameter. In another particular embodiment, the filtering unit comprises several layers of micro-beads having a size distribution of 15 μm to 25 μm diameter and several layers comprising micro-beads having a size distribution of 5 μm to 15 μm diameter. In a particular embodiment of the invention, each layer of micro-beads has a thickness ranging from 5 μm to 5 cm, 5 μm to 2 cm, from 5 μm to 1 cm, from 5 μm to 5 mm, from 5 μm to 2 mm, from 5 μm to 1 mm, from 5 μm to 500 μm, from 5 μm to 100 μm or from 5 μm to 10 μm. In another particular embodiment, each layer of micro-beads has a thickness ranging from 500 μm to 1 cm, preferably from 500 μm to 10 mm, still preferably from 1 mm to 10 mm, even preferably from 2 mm to 10 mm. In a more particular embodiment, each layer of micro-beads has a thickness ranging from 2 mm to 5 mm. In a particular embodiment, the layers of micro-beads have a total thickness ranging from 100 μm to 10 cm, from 500 μm to 5 cm, from 1 mm to 5 cm, from 2 mm to 5 cm, from 5 mm to 5 cm, from 1 cm to 5 cm or from 2 cm to 5 cm. The thickness and the number of layers within the filtering unit may be adjusted to take into account the amount of RBC containing blood product to be filtered. In the filtering unit, the layers of micro-beads are staked up on a filter suitable to maintain the beads and that is not involved in the retention capacity of the filtering unit. This support filter may be, for example, nylon filter or cellulose filter. Preferably, the pores of the support filter are from 5 to 25 μm diameter. Still preferably, the pores of the support filter are from 15 to 25 μm diameter. When the pores of the support filter have a diameter larger than the diameter of the smallest micro-beads, an additional layer of micro-beads can be poured onto the support filter to prevent leakage of the smallest micro-beads. This supporting layer is then between the filtering layer(s) and the support filter. Preferably, the diameter of micro-beads of this supporting layer is from 3 to 6 times larger than the smallest diameter of micro-beads in the filtering layer. In an embodiment, micro-beads of this supporting layer have a size distribution of 25 to 50 μm, still preferably of 25 to 45 μm. An additional support filter may be added between the supporting layer and the filtering layer(s) and/or between two filtering layers and/or above the last filtering layer to prevent resuspension of micro-beads of the previous layer when pouring micro-beads of the next layer or the red blood cell sample.

The flow through the filtering unit may be obtained using a syringe-pressured flow or by centrifugation (for example by centrifugating 1-2 minutes at 1500-2500 g). An electric pump, preferably a peristaltic pump, can be used to generate a constant flow, for instance a flow ranging from 1 mL/min to 1 L/min depending the size of the filtering unit. Preferably, the flow is ranging from 0.5 to 2 mL/min/gram of micro-beads in the filtering unit, preferably from 0.5 to 1.5 mL/min/gram. Alternatively, the flow through the filtering unit can also be obtained using any techniques known by the skilled person such as gravity-driven technique. In a particular embodiment of the invention, the filtration is performed under a constant pressure ranging from 30 to 90 cm of water (cmH₂O), preferably from 30 to 70 cmH₂O and more preferably from 30 to 50 cmH₂O. The filtration may be performed at a temperature ranging from 1° C. to 37° C., e.g. from 1° C. to 10° C. or from 1° C. to 6° C., preferably at a pressure of 30 to 50 Hectopascals.

The filtration of RBCs may be carried out by using a device comprising a filtering unit as disclosed above, a first compartment containing RBCs that are subjected to filtration, and a second compartment for receiving the filtrate, the filtering unit being in communication with the first and second compartments through conduit means, the first compartment being upstream of the filtering unit and the second compartment being downstream of the filtering unit. Optionally, the device may comprise means to generate a flow through the filtering unit, such as a pump or a syringe, preferably a peristaltic pump. The device may also be adapted to be centrifuged or elutriated. In an embodiment, the first compartment is directly connected to a vascular access of the donor. In an embodiment, the second compartment is directly connected to a vascular access of the recipient. In a particular embodiment, the filtering unit is placed between the first compartment and the vascular access of the recipient. The filtration device comprises at least one filtering unit as disclosed above. It may also comprise other filtering units comprising micro-bead layers as disclosed herein or any other types of filters. This device may be adapted on any device usually used to infuse a patient or on any apheresis device.

The filtrate obtained by filtration of RBCs through a filtering unit as disclosed above may be subjected to one or several other treatments before to be infused to a patient. The filtrate may be, for example, further subjected to leukoreduction filtration which decreases the transmission of leukocyte-associated viruses and HLA alloimmunization. Alternatively, the RBCs may be subjected to said additional treatment(s) before being subjected to filtration through micro-beads as disclosed above.

The present invention also concerns a method for preparing RBCs for infusion to a patient, which method comprises filtering RBCs through a filtering unit comprising one or several layers of micro-beads from 5 μm to 50 μm of diameter, preferably from 5 μm to 25 μm of diameter, recovering the filtrate, and obtaining RBC depleted of rigid red blood cells. All the embodiments of the RBC preparation of the invention are also contemplated in this method.

As used herein, the term “rigid RBCs” refers to a population of RBCs having an elongation index value less than 0.6. This elongation index value may be measured by any method known by the skilled person such as laser ektacytometry (i.e., LORCA system). As a reference, the elongation index value of fresh RBCs (collected for less than 24 hours) obtained from a human not affected by a RBC disorder, is in the range of 0.65 and 0.6.

The present invention also concerns a method for rejuvenation of RBCs for transfusion, which method comprises filtering said RBCs through a filtering unit comprising one or several layers of micro-beads from 5 to 50 μm, preferably from 5 to 25 μm, of diameters and recovering the filtrate, whereby obtaining a rejuvenated RBC preparation depleted of rigid red blood cells. All the embodiments of the RBC preparation of the invention are also contemplated in this method.

In a particular embodiment, RBCs are contained in whole blood and the method allows to obtain a rejuvenated blood for transfusion. In another particular embodiment, RBCs are packed RBCs and the method allows to obtain rejuvenated packed RBCs for transfusion.

The present invention further concerns a method for administering RBCs to a patient, which method comprises providing RBCs, filtering said RBCs through a filtering unit comprising one or several layers of micro-beads from 5 to 50 μm, preferably from 5 to 25 μm, of diameter, recovering the filtrate and infusing the patient with said filtrate. All the embodiments of the RBC preparation of the invention are also contemplated in this method.

The present invention also concerns a method for treating a patient having an inherited or acquired RBC disorder, which method comprises collecting blood from the patient, filtering said blood through a filtering unit comprising one or several layers of micro-beads from 5 to 50 μm, preferably from 5 to 25 μm, of diameter, recovering the filtrate and infusing the patient with said filtrate.

In an embodiment, the method is carried out during an erythrocytapheresis. In this case, the RBC fraction of the erythrocytapheresis is filtered through the micro-bead layers and the filtrate is infused to the patient with the other erythrocytapheresis fractions (plasma, platelet and leucocyte fractions). The filtrate may be infused to the patient as a mix with the other erythrocytapheresis fractions or may be simultaneously or sequentially infused with said fractions.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease.

The inherited or acquired RBC disorder which can be treated by the present method is a disorder inducing an altered deformability of RBCs, i.e. a decrease of the deformability. The filtration of the blood of the patient allows to retain rigid RBCs and thus to infuse him with a filtrate containing all blood components except rigid RBCs. The inherited or acquired RBC disorder which may be, for instance, hereditary or inherited spherocytosis, elliptocytosis (also named ovalocytosis), sepsis, hemoglobinopathy such as alpha or beta thalassemia, sickle cell disease, sickle cell trait (or sicklemia), diabetes mellitus, hemolytic anaemia such as auto immune hemolytic anaemia, and red blood cell enzyme deficiency such as glucose-6-phosphate deshydrogenase or pyruvate kinase deficiency. In a preferred embodiment, the patient is affected by sickle cell disease. In this case, depletion of rigid RBCs may be used to prevent acute or chronic complications related to the circulation of such RBCs with altered deformability, such stroke, chronic pain, or acute splenic or pulmonary sequestration.

The inherited or acquired RBC disorder may also be an infection with a protozoan parasite of the genus Plasmodium or Babesia. In this case, the depletion of rigid RBCs may be used to decrease the parasitic load of the patient. This treatment may be used in addition to antiparasitic drug administration. In an embodiment, the parasite is of the genus Plasmodium and may be chosen, for example, from the group consisting of Plasmodium falciparum, Plasmodium vivax, Plasmodium ovale, Plasmodium malariae, Plasmodium knowlesi, Plasmodium inui, Plasmodium cynomolgi, Plasmodium simiovale, Plasmodium brazilianum, Plasmodium schwetzi and Plasmodium simium, preferably from the group consisting of Plasmodium falciparum, Plasmodium vivax, Plasmodium ovale, Plasmodium knowlesi and Plasmodium malariae, and more preferably from the group consisting of Plasmodium falciparum and Plasmodium vivax. In another embodiment, the parasite is of the genus Babesia and may be chosen, for example, from the group consisting of Babesia microti, Babesia divergens, Babesia duncani, Babesia bovis, Babesia canis and Babesia bigemina.

All the embodiments of the RBC preparation of the invention are also contemplated in this method.

The following examples are given for purposes of illustration and not by way of limitation.

### EXAMPLES

### Example 1

Materials and Methods

Beads and Filtering Process:

Calibrated metal beads (96.50% tin, 3.00% silver and 0.50% copper) (Industrie des poudres Sphériques, Annemasse, France) with 2 different size distributions (5-15 μm diameter and 15-25 μm diameter) were used throughout.

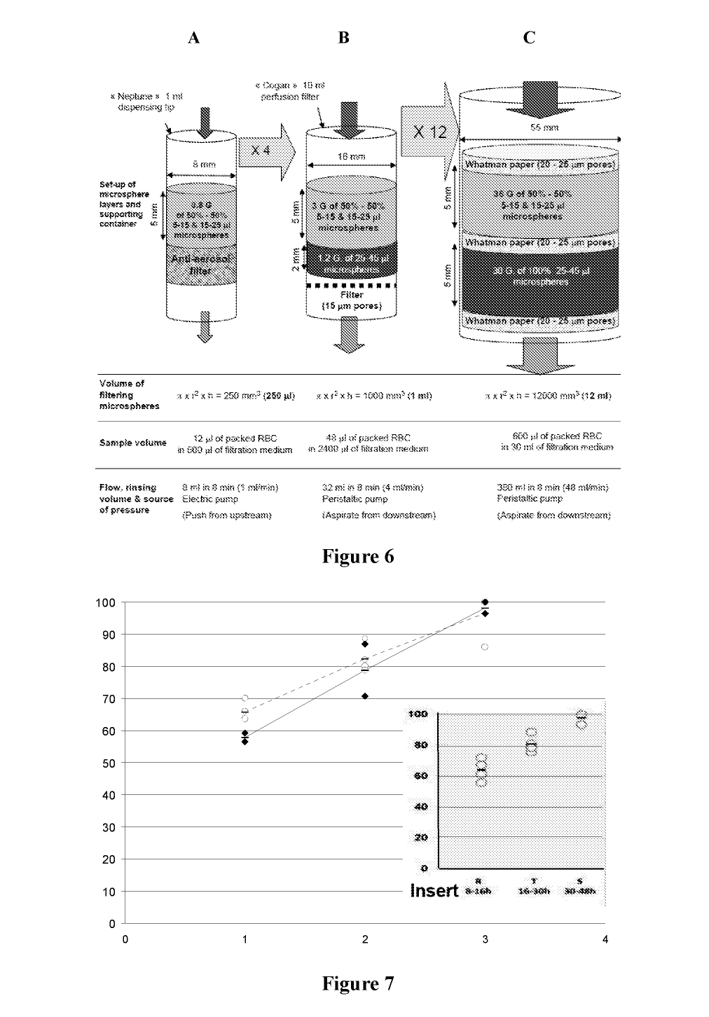

Two grams of dry beads of each sort were mixed and then suspended in 8 mL of PBS/1% AlbuMAX II®. 600 μl of this bead suspension was poured into an inverted 1000 μl anti-aerosol pipette tip (Neptune, BarrierTips) and allowed to settle, leading to the formation of a 5 mm-thick bead layer above the anti-aerosol filter. This filtering system is illustrated in FIGS. 2 and 6A. 600 μL of a 2% hematocrit RBC suspension containing less than 10% of potentially “retainable” RBCs was introduced upstream from the bead filter. RBCs were slowly flushed through the bead layer at a flow rate of 60 mL/hour using an electric pump (Syramed μsp6000, Arcomed'Ag). The bead layer was then washed with 8 mL of PBS/1% AlbuMAX II®. The downstream sample was retrieved. RBCs retained in the bead layer at the end of the entire procedure (filtration and washing steps) were separated from the beads by 3 successive settling steps. The concentration of RBCs in upstream, downstream and retained RBC samples was determined by examination of Giemsa-stained blood films or counting the percentage of PKH-labeled RBCs in cell suspensions.

Parasite Culture:

The FCR3 strain (FUP/CB line) (Fandeur et al., 1992) of P. falciparum was used throughout the experiments. Parasites were cultured using the method described by Trager and Jensen (Trager and Jensen, 1976) with the modifications described in Ralph et al. (Ralph et al., 2005). Cultures were tightly synchronized by lysing maturing forms through treatment with 0.3 M alanine at two successive cycles, starting 3 hours after ring-RBC appeared in the culture (Ralph et al., 2005). Parasites were harvested at different stages of development (ring-RBC: 0-8 h and 8-16 h; trophozoites: 16-30 h; schizonts: 30-48 h).

RBC Suspensions and PKH67-Labelling:

200 μl packed RBCs were washed twice in RPMI1640. Approximately 2.10⁹ washed cells were stained with PKH67 (Sigma, Deisenhofen, Germany) according to the manufacturer's instructions. In brief, after the removal of the RPMI medium, the cells were then suspended in 1.8 ml of diluent C (PKH67 Flurorescent Cell Linker Kit; Sigma), immediately mixed with 2 ml of a 1/500 dilution of PKH67 stock solution in diluent C and gently rocked at room temperature for 1.5 minutes. The cell suspension was then gently rocked for 1 minute, after which 4 ml of RPMI 2% AlbuMAX II® (Invitrogen) were added. RBCs were pelleted, transferred to a fresh centrifuge tube and washed twice in PBS/1% Albumax II®. Labeled RBCs were diluted in a ratio of 1 to 10 with unlabeled RBCs. A 2% hematocrit RBC suspension in PBS/1% AlbuMAX II® was prepared with this cell mix.

Heated Red Blood Cells:

RBCs were heated at 50° C. during 20 min after which they were PKH67-labeled. Labeled RBCs were mixed in a ratio of 1 to 10 with control unlabeled RBCs. A 2% hematocrit RBC suspension in PBS/1% AlbuMAX II® was prepared with this cell mix.

Hereditary Spherocytosis RBC:

RBCs from hereditary spherocytosis patients were obtained in the context of patients follow-up as approved by the Ile-de-France VI Institutional Review Board. RBCs were either studied by LORCA or labeled with PKH67 and then mixed in a ratio of 1 to 10 with normal unlabeled RBC. A 2% hematocrit RBC suspension in PBS/1% AlbuMAX II ® was prepared with this cell mix, and then filtered as described.

LDH Measurement:

LDH was quantified using Dimension® (Dade Behring, clinical chemistry system) according to the manufacturer's instructions.

Depletion of Rigid RBC from Stored Blood:

Deleucocytized blood was stored at 4° C. during 42 days under the same conditions as blood stored for transfusion with conservative medium (0.87% sodium chlorure, 0.017% adenine, 0.9% dextrose monohydrate, 0.52% mannitol) (Luten et al., 2008). Deformability of the RBC population and its retention rate in micro-bead filters were determined at weekly intervals from stored blood as described (see Heated RBC section). Fresh blood (collected the day of each experiment) was used as a control.

RBC deformability was measured by ektacytometry using a laser-assisted optical rotational cell analyzer (LORCA; Mechatronics, Hoorn, The Netherlands) as previously described (Hardeman and Ince, 1999). Elongation index (the unit of RBC deformability) was defined as the ratio between the difference between the two axes of the ellipsoid diffraction pattern and the sum of these two axes.

Results

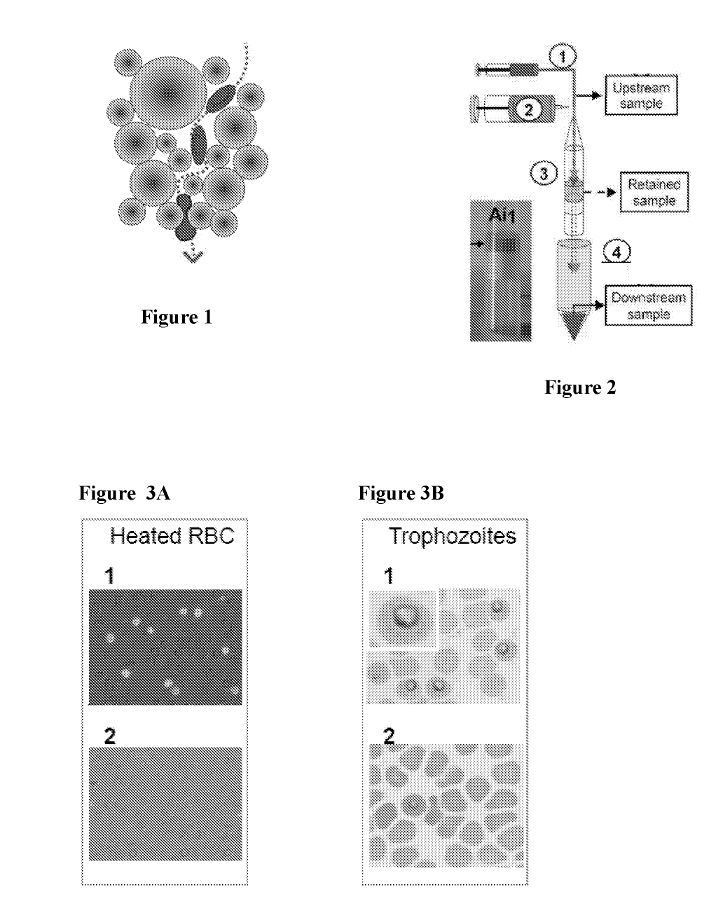

The filtering device comprised a mixture of 5-25 μm diameter beads mimicking the geometry of inter-endothelial slits of the splenic red pulp as described in the international patent application WO 2009/144586. This mixture generates narrow apertures (when 3 small beads are adjacent) interspersed with wider apertures (FIG. 1). The designed bead-filtering device consisted of a 5 mm-thick micro-bead layer deposited above the anti-aerosol filter of a tip (FIG. 2). Bead diameter heterogeneity led to the existence of a wide range of aperture sizes contributing to prevention of clogging of the filtering device.

The system was first evaluated with RBCs incubated for 20 min at 50° C., a treatment known to impair their deformability and induce their spleen clearance when injected in vivo (Atkins et al., 1980). Retention rates for heated RBC were consistently above 95% (FIG. 3A). Three technical replicates led to similar results, highlighting the excellent reproducibility of the technique.

Retention rates for RBCs collected from patients with hereditary spherocytosis (HS) were also measured and were ranging from 44% to 88%. Interestingly, the retention rate of HS-RBC in micro-beads correlated with the elongation index determined by LORCA (Spearmann correlation coefficient R²=0.96).

Retention rates for RBCs infected with Plasmodium falciparum obtained from in vitro culture were also assessed. It was observed that micro-bead retention rates of Pf-RBC (RBC infected by P. falciparum) correlated with parasite maturation, with a mean (SD) retention rate of 46.0% (1.4), 64.2% (7.0), 81% (5.6), and 97.8% (3.8) for young ring-RBC (0-8 h), late ring-RBC (8-16 h), trophozoites (FIG. 3B), and schizonts, respectively.

Filtering-induced haemolysis was estimated by measuring LDH released in the supernatant of the flow through sample, and was consistently below 3% for both heat-treated and HS-RBCs. Importantly, there were no schizocyte, dacryocyte or poikilocyte downstream from the micro-beads, contrasting with filtration through narrow polycarbonate sieves (2 μm-wide, 20 μm-long channels). This indicated that, despite its ability to retain rigid RBCs, the micro-bead filtration process did not induce cell fragmentation and preserved RBC morphology (FIG. 5).

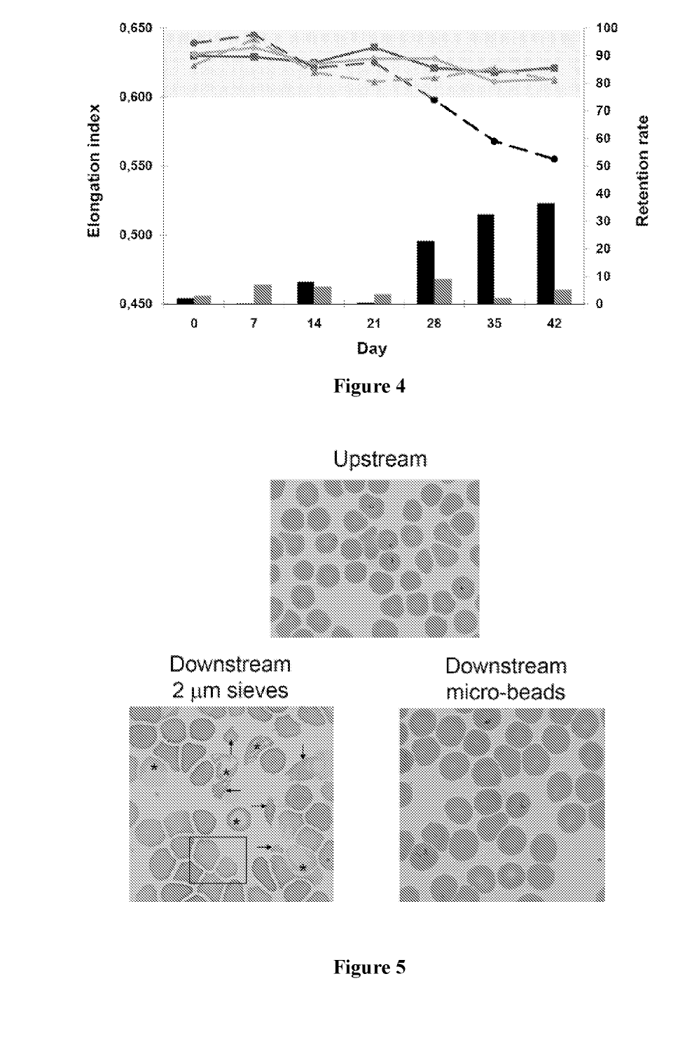

Prolonged storage of RBC concentrates is associated with a decrease of the RBC deformability and with an increase in the incidence of several transfusion-related side-effects. To evaluate the potential of micro-beads to filter out rigid RBCs from stored RBC concentrates and thereby improve the quality of the transfused blood, deformability of RBCs kept under blood bank storage conditions for 6 weeks was studied using the LORCA methodology. The rigid RBC clearance capacity of micro-bead filters and the morphology of RBC in the filtrate were also studied. In two independent experiments, the inventors observed that the elongation index of RBC concentrates decreased significantly after 3 weeks of storage (FIG. 4). A parallel increase of the retention rate of stored RBC in micro-beads was observed (FIG. 4). No retention was detected with RBC stored for 3 weeks or less, while 30.6% of RBC stored for 3-6 weeks were retained by the filter. These results matched with the 26.5% 24-hour clearance rate of RBC transfused to patients after a 25 to 35-day storage period (Luten et al., 2008). Importantly, the elongation index of the stored RBC population was restored to normal value after filtration through micro-beads (FIG. 4).

### Example 2

Materials and Methods

Parasite culture, preparation of RBC suspension, PKH67-labelling and preparation of heated RBCs were done as described in example 1.

Beads and Filtering Process:

Filtering layers comprised calibrated metal beads (96.50% tin, 3.00% silver and 0.50% copper) (Industrie des poudres Sphériques, Annemasse, France) with two different size distributions (50% (w/w) of micro-beads having a size distribution of 15 μm to 25 μm diameter and 50% (w/w) of micro-beads having a size distribution of 5 μm to 15 μm diameter).

Supporting layers comprised calibrated metal beads as described above with a size distribution of 25 μm to 45 μm diameter.

Filtering Systems

1. The infusion set/bubble trap system: (Sterile 175 cm infusion set, LL 86-P ISO 8536-8-IS-P, CODAN Medizinische Geräte GmbH & Co KG Stig Husted-Andersen Straβe 11, D-23738 Lensahn, Germany). This filtering system is illustrated in FIG. 6B. The filter at the bottom of the bubble trap is made of 15 μm-wide pores in a membrane. The perfusion kit was cut at the upper part of the 8-ml bubble trap. Because micro-beads (or microspheres) smaller than 15 μm in the filtering 5-25 μm micro-bead mixture would not be retained by the 15 μm pores in the membrane, a supporting 2 mm-thick layer of 25-45 μm micro-beads was deposited above the membrane. The supporting layer was created by pouring into the bubble trap 1.2 g of the 25-45 μm micro-bead mixture previously resuspended in 1 ml of PBS-albumin. Once this first layer had sedimented, the true filtering layer of 5-25 μm micro-beads was prepared by gently pouring 3 g of the mixture in 4 ml of PBS albumin. Once this second layer had sedimented, tubing downstream from the bubble trap was adjusted into the peristaltic pump (2-way Minipuls 3, Gilson M312), and the extremity was introduced into the collecting 15-ml tube. 2.4 ml of the red blood cell suspension (at 2% hematocrit) containing less than 10% of abnormal red blood cells was introduced in the bubble trap. The peristaltic pump was then switched on to generate a 4 ml/min flow. During the filtration process 24 mL of PBS/AlbuMAX II were gently added into the bubble trap for a final rinsing volume of 32 ml.

2. The “Filtron” device: This filtering system is illustrated in FIG. 6C. Two 55-mm diameter filter papers with pore size of 20-25 μm (N°4 qualitative circles Cat No 1004-055, Whatman International Ltd Maidstone, England, UK) were deposited at the bottom of the device—a 200-ml cylinder with a grid at the bottom and a single way-out connector downstream from the grid. A 4 mm-thick supporting micro-bead layer was then created above the filter paper by gently pouring into the device 30 g of 25-45 μm micro-beads, resuspended in 50 ml of PBS-albumin. A filter paper was deposited at the top of the 25-45 μm micro-bead layer once it had sedimented, to avoid resuspension of the micro-beads while pouring the second, filtering layer of 5-25 μm micro-beads. This second layer was prepared by gently pouring 36 g of the mixture (previously resuspended in 50 ml of PBS albumin) into the device. Once this second layer had sedimented, a filter paper was deposited at the top of the 5-25 μm micro-bead layer. Tubing downstream from the device was adjusted into a peristaltic pump (ISM 920 pump from Ismatec, Labortechnik, Glattbrugg-Zurich, Switzerland; and Masterflex 06419-17 tube from Tygon, Saint-Gobain, France), and the extremity of tubing was introduced into the collecting 50-ml tubes. 30 ml of the red blood cell suspenion (at 2% hematocrit) containing less than 10% of abnormal red blood cells was introduced in the device. The peristaltic pump was then switched on to generate a 48 ml/min flow. During the filtration process 300 mL of PBS/AlbuMAX II were gently added into the device for a final rinsing volume of 380 ml.

For each of these two filtering systems, up- and down-stream samples were collected, centrifuged 5 min at 1500 g. The proportion of parasitized red blood cells was quantified on Giemsa-stained smears as described in example 1.

Results

As shown on FIG. 7, retention rates of RBCs infected with Plasmodium falciparum at different stages (rings, trophozoites and schizonts) were similar with filtering systems comprising from 250 μL to 12 mL of filtering micro-beads and with sample volumes from 600 μL to 30 mL. An approximately 50-fold increase in the sample volume thus does not alter the performance of the filtration. These results demonstrate the feasibility of scaling-up the RBC filtration process.

Conclusion

These results demonstrate that RBCs filtration on micro-beads allows the clearance of rigid RBCs while preserving the morphology and integrity of RBCs. This micro-bead filtration is thus suitable to obtain RBC preparation depleted of rigid RBCs from RBCs that have been previously stored, heated or from RBCs obtained from patients affected by RBC disorders or infections inducing a decreased deformability of RBC. This RBC preparation can thus be infused to any patient in need thereof.

### REFERENCES

Atkins, H. L., et al. Splenic sequestration of 99mTc labeled, heat treated red blood cells. Radiology 136, 501-503 (1980).

Fandeur, T., Gysin, J. & Mercereau-Puijalon, O. Protection of squirrel monkeys against virulent Plasmodium falciparum infections by use of attenuated parasites. Infect Immun 60, 1390-1396 (1992).

Hardeman M R, Ince C. Clinical potential of in vitro measured red cell deformability, a myth? Clin Hemorheol Microcirc. 21(3-4):277-84. (1999)

Huruta, R. R., et al., Mechanical properties of stored red blood cells using optical tweezers. Blood, 1998. 92(8): p. 2975-7.

Izzo, P., et al., Erythrocytes stored in CPD SAG-mannitol: evaluation of their deformability. Clin Hemorheol Microcirc, 1999. 21(3-4): p. 335-9.

Kirkpatrick, U. J., et al., Rheological properties and function of blood cells in stored bank blood and salvaged blood. Br J Haematol, 1998. 101(2): p. 364-8.

Koch, C. G., et al., Duration of red-cell storage and complications after cardiac surgery. N Engl J Med, 2008. 358(12): p. 1229-39.

La Celle, P. L., Alteration of deformability of the erythrocyte membrane in stored blood. Transfusion, 1969. 9(5): p. 238-45.

Luten, M., et al., Survival of red blood cells after transfusion: a comparison between red cells concentrates of different storage periods. Transfusion, 2008. 48(7): p. 1478-85.

Mohandas, N. and P. G. Gallagher, Red cell membrane: past, present, and future. Blood, 2008. 112(10): p. 3939-48.

Mohandas, N., W. M. Phillips, and M. Bessis, Red blood cell deformability and hemolytic anemias. Semin Hematol, 1979. 16(2): p. 95-114.

Nash, G. B., et al., Abnormalities in the mechanical properties of red blood cells caused by Plasmodium falciparum. Blood, 1989. 74(2): p. 855-61.

Ralph, S. A., et al. Transcriptome analysis of antigenic variation in Plasmodium falciparum—var silencing is not dependent on antisense RNA. Genome Biol 6, R93 (2005).

Stadler, I., C. A. Toumbis, and J. L. Ambrus, Influence of cold storage altered red cell surface on the function of platelets. J Med, 1994. 25(6): p. 353-61.

Trager, W. & Jensen, J. B. Human malaria parasites in continuous culture. Science 193, 673-675 (1976).

1-21. (canceled)

22. A method for administering red blood cells (RBCs) to a patient, which method comprises providing RBCs, filtering said RBCs through a filtering unit comprising one or several layers of micro-beads from 5 to 50 μm of diameter, recovering the filtrate and infusing the patient with said filtrate.

23. The method according to claim 22, wherein the filtering unit comprises one or several layers of micro-beads from 5 to 25 μm of diameter.

24. The method according to claim 22, wherein the RBCs that are subjected to filtration are whole blood or a RBC-containing fraction.

25. The method according to claim 22, wherein the RBCs that are subjected to filtration have been previously stored.

26. The method according to claim 22, wherein the RBCs that are subjected to filtration have been obtained from a patient having an inherited or acquired red blood cell (RBC) disorder.

27. The method according to claim 26, wherein the inherited or acquired RBC disorder is selected from the group consisting of hereditary or inherited spherocytosis, elliptocytosis, sepsis, hemoglobinopathy, sickle cell disease, sickle cell trait, diabetes mellitus, hemolytic anaemia, and a RBC enzyme deficiency.

28. The method according to claim 27, wherein the inherited or acquired red blood cell disorder is sickle cell disease.

29. The method according to claim 22, wherein the RBCs that are subjected to filtration are a RBC concentrate.

30. The method according to claim 22, wherein the RBCs that are subjected to filtration are a RBC fraction of erythrocytapheresis.

31. The method according to claim 22, wherein the RBCs that are subjected to filtration are obtained from a human.

32. The method according to claim 22, wherein the RBCs that are subjected to filtration are obtained from the patient to be infused.

33. The method according to claim 22, wherein the RBCs that are subjected to filtration are obtained from a blood bank.

34. The method according to claim 22, wherein at least one layer of micro-beads of the filtering unit is constituted by a mix of micro-beads having a size distribution of 15 to 25 μm diameter and micro-beads having a size distribution of 5 to 15 μm diameter.

35. The method according to claim 34, wherein at least one layer of micro-beads of the filtering unit is constituted by a mix of micro-beads having a size distribution of 15 μm to 25 μm diameter and micro-beads having a size distribution of 5 μm to 15 μm diameter and comprises at least 50% (w/w) of micro-beads having a size distribution of 5 μm to 15 μm diameter.

36. The method according to claim 35, wherein said at least one layer of micro-beads is constituted by about 50% (w/w) of micro-beads having a size distribution of 15 μm to 25 μm diameter and about 50% (w/w) of micro-beads having a size distribution of 5 μm to 15 μm diameter.

37. The method according to claim 22, wherein each layer of micro-beads has a thickness selected from 500 μm to 1 cm, 500 μm to 10 mm, 1 mm to 10 mm, or 2 mm to 10 mm.

38. A method for preparing RBCs for infusion to a patient, which method comprises filtering RBCs through a filtering unit comprising one or several layers of micro-beads having a diameter of 5 to 50 μm or 5 to 25 μm, recovering the filtrate, and obtaining RBCs depleted of rigid red blood cells.

39. A method for rejuvenation of RBCs for transfusion, which method comprises filtering said RBCs through a filtering unit comprising one or several layers of micro-beads having a diameter of 5 to 50 μm or 5 to 25 μm and recovering the filtrate, whereby obtaining a rejuvenated RBC preparation is depleted of rigid red blood cells.

40. A method for treating a patient having an inherited or acquired RBC disorder, which method comprises collecting blood from the patient, filtering said blood through a filtering unit comprising one or several layers of micro-beads having a diameter of 5 to 50 μm or 5 to 25 μm, recovering the filtrate and infusing the patient with said filtrate.

41. The method according to claim 40, wherein the inherited or acquired RBC disorder is selected from the group consisting of hereditary or inherited spherocytosis, elliptocytosis (also named ovalocytosis), sepsis, hemoglobinopathy, alpha or beta thalassemia, sickle cell disease, sickle cell trait (or sicklemia), diabetes mellitus, hemolytic anaemia, auto immune hemolytic anaemia, red blood cell enzyme deficiency, glucose-6-phosphate deshydrogenase deficiency, pyruvate kinase deficiency, and an infection with a protozoan parasite of the genus Plasmodium or Babesia.
